(19) United States
(12) Patent Application Publication
(10) Pub. No.: US 2005/0065175 A1
(43) Pub. Date: Mar. 24, 2005

(54) Oral transmucosal methadone

(75) Inventors: Gilbert R. Gonzales, New York, NY (US); Paolo L. Manfredi, New York, NY (US)

Correspondence Address: WOOD, HERRON & EVANS, LLP, 2700 CAREW TOWER, 441 VINE STREET, CINCINNATI, OH, 45202, US

(73) Assignee: Xanodyne Pharmacal, Inc.

(21) Appl. No.: 10/669,490

(22) Filed: Sep. 24, 2003

(51) Int. Cl. A61K 31/485; A61K 9/14; A61K 9/68

(52) U.S. Cl. 514/282; 424/48; 424/440

(57) ABSTRACT

Pharmaceutical compositions for oral-transmucosally administering methadone. For oral transmucosal delivery, the compositions comprise an oral dissolution agent and methadone in a dosage formulation. The dissolution agent generally causes the formulation to break down or dissolve in the oral cavity. The formulation is buffered to a pH of at least 6 for substantial absorption of the methadone through the oral mucosal tissue from the dissolved dosage form.

### BACKGROUND OF THE INVENTION

I. Field of the Invention

The invention is generally related to the administration of methadone and, in particular, to the administration of methadone through oral mucosal tissue.

II. Description of the Prior Art

Opioids, in general, are effective for treating a wide spectrum of painful conditions. For example, opioids are commonly administered for treatment of neuropathic pain, cancer pain, and other chronic pain conditions, such as arthritic pain. Neuropathic pain is the predominant mechanism in patients with chronic pain syndromes such as those suffering from post-herpetic neuralgia and peripheral neuropathy, and is also a contributor to cancer pain. More recently, opioid administration for the treatment of neuropathic pain has increased.

One advantage of opioid treatment is that its toxicity is generally limited to specific, well known, manageable side effects. More importantly, opioids are generally non toxic with respect to body organs. However, there are two main limitations to the use of opioids, particularly in treating pain: (1) With chronic opioid use, patients generally have become tolerant to the analgesic effects and have required escalation in doses to maintain desired analgesic effects. Such dosage escalations have resulted in increased side effects, such as sedation and constipation; and (2) some types of pain are generally less responsive to opioid analgesics, for example, neuropathic pain is not adequately treated with many known opioid analgesics.

Methadone is an opioid analgesic with a unique, therapeutic profile. More specifically, methadone is a mu receptor agonist commonly compared with the standard opioid analgesic, morphine, in terms of efficacy and application. Aside from its agonistic activity at the mu receptor, methadone has other actions that are thought to contribute to its unique analgesic profile. Particularly, methadone exerts antagonistic activity at the N-methyl-d-aspartate (NMDA) receptor. Methadone's activity at the NMDA receptor has been shown to counteract opioid tolerance in experimental models of pain. Such an antagonistic effect is thought to be the basis by which methadone is as effective or more effective than morphine, even with only a minimal dose escalation over time. Methadone's ability to antagonize the NMDA receptor also results in increased efficacy in treating hyperalgesia while enhancing its effectiveness for treating neuropathic pain and other chronic pain states. Accordingly, methadone's antagonistic activity at the NMDA receptor is believed to be the primary reason for its superior analgesic effects. Structurally, both the d-isomer and the I-isomer of methadone antagonize the NMDA receptor. However, only the I-isomer is thought to be primarily responsible for the mu receptor agonistic effect.

There is also experimental evidence that methadone's therapeutic effects from binding at mu opioid receptors may be broader than the effects of other opioids binding at the same receptor sites. This is particularly important, as clinical experience has revealed that patients who do not respond to one opioid, and/or develop intolerable side effects before experiencing pain relief from the opioid, may instead respond to another opioid. This clinical phenomenon may be partly determined by individual genetic differences in opioid receptor subtypes density and distribution. Patients who have been on other opioid analgesics, and later rotated to methadone therapy, have exhibited improved pain relief. Such a rotation may be due to methadone's postulated broader spectrum of activity at the opioid receptor sites. Moreover, tolerance to the analgesic effects of methadone develops more slowly than with other commonly used opioids. In addition, methadone has an extended duration of action in suppressing withdrawal symptoms in physically dependent individuals and, in particular, the withdrawal signs and symptoms occurring after abrupt discontinuance of methadone are milder than those of morphine.

Thus, methadone is an efficacious and useful medication. Methadone is an effective analgesic, generally administered for the treatment of pain, especially pain refractory to other medications. Methadone is effective for the relief and management of severe, constant pain, such as chronic cancer pain, and many other types of pain, including neuropathic pain. Particularly, methadone is known to inhibit the re-uptake of both norepinephrine and serotonin. Medications that share this effect have, in the past, been the backbone of the treatment of neuropathic pain. Thus, this is another mechanism by which methadone has been effective in treating pain. Methadone is also administered for treatment of opioid abstinence syndromes, and for the treatment, detoxification, and maintenance of a chronic, relapsing drug addict and, in particular, a heroin addict. Methadone has also been found to be an effective antitussive agent.

Currently available formulations of methadone include methadone hydrochloride tablets, U.S.P., available for oral use in 2.5, 5 mg , 10 mg, and 40 mg amounts. Methadone hydrochloride, U.S.P. is a bitter, white powder, soluble in water and ethanol, and the tablets are dosed by oral ingestion (absorption into the blood through the gastrointestinal tract or gut). In an effort to prevent its unregulated, large-scale use, particularly in the treatment of opioid addiction, the Food and Drug Administration of the United States Government (FDA) has regulated methadone use for drug addiction to specialized dosage forms including tablets.

Such orally administered tablets provide methadone through the gastrointestinal (GI) tract, which can be detected in blood plasma typically within 30 minutes after ingestion. However, methadone absorbed in the gut undergoes extensive metabolism in the liver. In addition, methadone administered in such a manner results in a half-life of a single methadone dose of approximately 15 hours and, therefore, requires a large dose or multiple doses on a daily basis to achieve the desired effects.

Methadone has also approved by the FDA for parenteral administration. However, the only commercial formulation approved and available for use is a 20 ml multi-dose vial containing a solution of methadone (10 mg/ml) and a 0.5% chlorobutanol (5 mg/ml) as a preservative. It is believed that the presence of chlorobutanol, even in such small amounts, may be linked to the cause of deaths of patients who suffer from stage IV cancer and receive the FDA approved parenteral methadone solution via intravenous injection (IV).

Some opioid analgesics, such as fentanyl, have been administered through the oral mucosal tissue. However, for effective transmucosal absorption, the drug formulation must address problems associated with the oral environment. For example, there is generally a relatively small amount of solvent (saliva or other fluid) in the oral cavity into which the drug formulation can breakdown to deliver the drug. Particularly, the relative amounts of saliva produced in the oral cavity under any given circumstance can vary widely. On the average, salivary glands produce between about 800 to about 1500 mls of saliva per day. In a resting, unstimulated state, salivary glands produce about 0.5 ml mucous-type saliva per minute, while stimulated salivary glands, such as during chewing, biting, sucking, licking or other oral activity, produce about 1-3 mls/minute. Thus, in as little as about 10-15 minutes, the time typically required for oral delivery of a dosage of an average drug, the total amount of saliva produced in the oral cavity is about 10-15 mls when stimulated. The oral cavity, therefore, has a far smaller volume of “dissolving” fluid than the 600 to about 1000 mls of potentially degradative and dissolving solvent produced in the GI tract.

Similarly, an orally ingested drug has a far less “absorption” time period in the oral cavity than it has in the GI tract. A dosage orally-delivered to the GI tract usually remains in the GI tract for several hours, as compared to the same formulation generally remaining in the oral cavity no longer than a mere 10 to 15 minutes. In addition, the rate of the transmucosal absorption is dependent on the surface area available for drug absorption. The surface area in the oral cavity (about 200 cm²) is small relative to the surface area of other drug delivery routes, such as the GI tract (350,000 cm²) or the skin (20,000 cm²). Thus, during this brief period and with the small available mucosal surface area, the formulation should be capable of releasing or delivering the drug, and optimizing contact time with the absorption surface, for effective transmucosal absorption.

Because of methadone's unique pharmacodynamic properties and therapeutic effects, it is desirable to provide an administrable formulation of methadone that will effectively treat patients with pain and, in particular, pain refractory to other opioids, while exhibiting a decreased patient methadone tolerance profile. It is also desirable to provide methadone in a formulation sufficient to treat severe or “breakthrough pain” resulting from the incidence of cancer or cancer-related physiological ailments. It is also desirable to provide a formulation which provides methadone to the blood in a readily bioavailable and more efficacious manner than oral ingestion and/or parenteral administration. It is also desirable to decrease the amounts of methadone in the formulation while providing desired therapeutic effects. It is further desirable to provide a formulation that may be government-approved.

### SUMMARY OF THE INVENTION

The present invention provides pharmaceutical compositions, comprising methadone, designed to deliver the methadone to the body in a manner more efficacious than previously formulated, and approved, methadone-containing compositions. More specifically, the compositions provide methadone for absorption through the oral mucosal tissue of a patient. In one embodiment, the composition comprises a dosage formulation including an oral dissolution agent and methadone buffered to a pH of at least about 6 for substantial absorption of methadone through the oral mucosa from the dissolved dosage form. In this manner, the compositions deliver the methadone to the oral mucosal tissue for transmucosal absorption into the patient's bloodstream.

Transmucosal methadone provides all of methadone's benefits and advantages more quickly and more effectively than prior methadone-containing compositions. Transmucosal absorption of methadone is effective, with an absorption rate as high as about 75%. Because of its high liposolubility, methadone readily crosses mucous membranes, resulting in rapid transmucosal absorption, as compared to absorption through the GI tract or via intravenous or intramuscular routes. Methadone's liposolubility also contributes to high sublingual absorption, particularly at an alkaline pH. Accordingly, methadone is readily bioavailable when absorbed through the mucosal tissue and the therapeutic effects of methadone are generally experienced in a shorter period of time. For example, 50-70% of the orally bioavailable methadone is into the bloodstream within the first 2.5 minutes of delivery to the mucosal tissue. In addition, transmucosal absorption of methadone in the oral cavity overcomes drawbacks, such as local irritation, related to intranasal administration and/or subcutaneous administration. Further, oral transmucosal delivery of methadone eliminates the need for preservatives, such as chlorobutanol, thereby addressing the toxicity related to government-approved parenteral formulations. As such, pain relief obtained with transmucosal methadone provides many benefits over other routes of administration to a patient.

The composition includes therapeutically effective amounts of methadone. In one embodiment, methadone is present in a dosage of at least about 0.5 mg. In another embodiment, it is present in a range from about 2 mg to about 50 mg per dose. The dosage generally depends upon the targeted patient and the particular formulation. For example, amounts in the range from about 2 mg to about 6 mg are generally suitable in children while higher amounts, such as in the range from about 6 mg to about 25 mg, are generally suitable for adults.

The oral dissolution agent allows the formulation to deliver a substantial portion of its methadone to the oral mucosal tissue in the oral cavity. In one embodiment, the oral dissolution agent includes at least one of acacia, alginic acid, carbomer, carboxymethylcellulose, calcium, carboxymethylcellulose sodium, microcrystalline cellulose, dextrates, dextrin, dextrose, methyl cellulose, ethyl cellulose, fructose, gelatin, guar gum, hydroxyethyl cellulose, hydroxypropyl cellulose, hydroxypropyl methylcellulose, lactitol, lactose, lecithin, maltodextrine, mannitol, poloxamer, polyethylene glycol, polymethacrylates, polyoxyethylene alkyl ethers, polyvinyl alcohol, propylene glycol alginate, sodium alginate, sodium ascorbate, sodium starch glyolate, sodium saccharin, sorbitol, starch, pre-gelatinized starch, sucrose, tragacanth, trimethylglycine, xanthan gum, xylitol, and zein to enhance solubility and dissolution of the composition in the oral cavity.

The pH of the oral environment affects the rate of transmucosal absorption of the methadone. To this end, the composition is buffered to a pH of at least about 6. At a mildly acidic pH in the range from about 6.5 to about 6.9, typically that of saliva of a normal patient, methadone is about 34% bioavailable via the oral transmucosal route. However, as the pH of the oral cavity, and the saliva in particular, increases to a pH in a range from about 7 (neutral) to about 10 (alkaline), bioavailability of the methadone via the oral mucosa increases, and has been measured as high as about 75% at a pH of about 8.5. In another embodiment, the composition further includes a pH buffer, such as a phosphate buffer, a glycylglycine buffer, a carbonate buffer, a bicarbonate buffer, a tris buffer, a tartrate buffer, a borate buffer, an acetate buffer, a maleate buffer or a combination thereof, to buffer the oral environment to a desired pH or pH range. The buffer should be present in an amount suitable to provide the desirable pH in an average patient's oral cavity upon administration of the composition.

The components of the composition may be combined in any suitable formulation so as to deliver a substantial portion of the methadone to the mucosal tissue in the oral cavity. Suitable formulations include, without limitation, solid formulations such as lozenges, lollipops, troches, dragees, chewable gums, solid candies, granular solids such as powders, chewable tablets or pills, orally dispersable tablets or pills, orally dissolvable tablets, pills or capsules, and the like, as well as liquid or semi-solid formulations such as solutions, suspensions, pastes, creams, lotions, and emulsions. The composition may also include other desirable components, such as commonly used excipients.

The compositions are useful for treating many indications and, in particular, for the treating pain. For example, the compositions may be used to treat “breakthrough pain”, that is, episodes of moderate to severe pain lasting a few minutes to several hours, that occur on a background of well controlled pain. In one embodiment, the compositions are used to treat pain attributed to one of cancer pain, neuropathic pain, chronic pain, acute pain, somatic pain, autonomic nervous system mediated pain, central pain, post-herpetic neuralgic pain, and combinations thereof. In another embodiment, the compositions are use to treat drug addiction, opioid tolerance, pathological itching, seizure, a sedative effect, a euphoric effect, an antitussive effect, an NMDA antagonistic effect, an opioid substitute for reducing opioid induced constipation, and a reduction of at least one of catacholamine uptake, norepinephrine re-uptake and serotonine re-uptake, and a combination thereof in the patient.

These and other advantages and benefits of the present invention will be further appreciated in light of the following detailed description of exemplary embodiments.

### DETAILED DESCRIPTION OF EXEMPLARY EMBODIMENTS

The present invention provides compositions comprising transmucosal methadone formulations. The formulations comprise methadone, an oral dissolution agent and a buffer. The methadone should be present in therapeutically effective amounts. Generally, the composition comprises methadone in a dosage of at least about 0.5 mg. In one embodiment, the composition comprises methadone in a dosage ranging from about 2 mg to about 50 mg. In another embodiment, the composition comprises methadone in an amount ranging from about 2 mg to about 10 mg per dose. The precise amount of the methadone generally depends upon many factors, such as age, size, weight, gender and medical history of the targeted patient population, the particular formulation, as well as the purpose of administration. Amounts will be further discussed herein with respect to administration of the composition.

The physical and chemical properties of the methadone, i.e., the particular form of methadone such as a water soluble salt or a hydrophobic, water insoluble free-base, generally affects the rate of transmucosal absorption. For example, the lipophilic, free-base form more readily diffuses across the mucosal membrane than an ionized or salt form. However, a buffer, discussed herein, may serve to deliver the methadone, initially formulated as a salt, as its free-base form to the mucosal tissue. The form of the methadone influences the solubility and dissolution of the methadone in the fluids of the oral cavity, which are two aspects in creating a positive concentration gradient across the oral mucosa. Particularly, a higher concentration in the oral cavity than in the blood is the driving force for absorption into the blood circulation.

The compositions further include an oral dissolution agent to enhance the delivery or release of the methadone from the dosage formulation to the surfaces of the oral mucosa. Suitable oral dissolution agents include, for example, commonly used and accepted pharmaceutical ingredients, such as sugars, saccharides, carbohydrates, polymers, excipients, and the like, capable of breaking down in and/or dissolving in fluids of the oral cavity. Examples of suitable oral dissolution agents include, without limitation, acacia, alginic acid, carbomer, carboxymethylcellulose, calcium, carboxymethylcellulose sodium, microcrystalline cellulose, dextrates, dextrin, dextrose, methyl cellulose, ethyl cellulose, fructose, gelatin, guar gum, hydroxyethyl cellulose, hydroxypropyl cellulose, hydroxypropyl methylcellulose, lactitol, lactose, lecithin, maltodextrine, mannitol, poloxamer, polyethylene glycol, polymethacrylates, poly oxyethylene alkyl ethers, polyvinyl alcohol, propylene glycol alginate, sodium alginate, sodium ascorbate, sodium starch glyolate, sodium saccharin, sorbitol, starch, pregelatinized starch, sucrose, tragacanth, trimethylglycine, xanthan gum, xylitol, zein, and combinations thereof.

The selection of the oral dissolution agent or agents is generally dependent on the form of the methadone, as well as on the process used in making the formulation and the intended use and characteristics of the formulation (e.g., taste for oral transmucosal delivery). The dissolution agent may be combined with the methadone in a suitable formulation. In this case, the dissolution agent and the methadone should be able to mix at the molecular level. For example, if the co-melt process is used to make a solid formulation, the dissolution agent should be capable of acting as a solvent into which the methadone can dissolve or melt. If a partial wet granulation process is used, the dissolution agent and the methadone should be able to dissolve in the proper solvent for this process.

The dissolution agent(s) may be selected to provide stability for the methadone. Where the dissolution agent is mixed with the methadone at the molecular level, it may serve as a physical barrier for preventing the methadone from being contacted by other ingredients, otherwise incompatible therewith, in the formulation or in the environment. For example, if the form of methadone can be degraded, via hydrolysis, with water, the use of a non-hygroscopic dissolution agent can prevent water from contacting and hydrolytically decomposing the methadone.

The amount of the dissolution agent(s) selected is generally dependent upon the formulation, its characteristics, and the purpose of administration. For example, where the formulation is a lozenge or a lollipop (lozenge on a stick) designed to be sucked on and/or licked, generally a majority of the formulation, such as up to about 99% by weight, may comprise the oral dissolution agent or combinations thereof. Other formulations, such as tablets, may include lesser amounts, such as between about 10% to about 80% by weight. The amount of the oral dissolution agent also determines the time period for which the methadone is exposed to the oral mucosa surface. For example, where the formulation is designed to remain in the oral cavity for only a short period of time and, therefore, must deliver at least a substantial portion of the methadone in that time, fast-dissolving or increased amounts of the dissolution agent(s) would be desirable. On the other hand, where the formulation includes larger dosages of methadone and/or a higher concentration of the methadone in the oral cavity is required for substantial absorption through the mucosa, then less amounts of the dissolution agent(s) may be suitable. To provide a longer duration of absorption for the methadone, decreased amounts and/or slower-dissolving oral dissolution agents may be suitable. In any event, the oral dissolution agent(s) will generally affect the rate of delivery of the methadone to the oral mucosa and, therefore, the rate of absorption, efficacy, and duration of action of the transmucosally administered methadone.

The rate of absorption of methadone by the oral mucosa also depends upon the environment in which the methadone is absorbed across the mucosal tissue. More specifically, the pH of the oral cavity during absorption affects the rate of the absorption. Accordingly, the compositions are formulated at a pH of at least about 6. Manipulating the oral cavity's pH through the formulation can affect the rate of absorption as well as the overall therapeutic effect of the methadone. For example, the solubility of methadone in the oral environment, and particularly in saliva, can be increased by buffering the pH in the oral cavity to an acidic value at which the methadone is likely to be in an ionized form. However, ionization may also limit rate of diffusion of the methadone through the mucosa by decreasing the lipophilicity of the methadone.

To this end, the composition advantageously includes a pH buffer. A pH buffer is a substance or system that is generally capable of maintaining a solution or a medium within a particular pH range, or at a particular pH. A buffer system generally includes a hydrogen ion donor(s) (acid) and conjugate hydrogen ion receiver(s) (base). Thus, a pH buffer serves to regulate the pH in the oral cavity for transmucosal absorption of the methadone. For example, normal saliva has a mildly acidic pH of about 6.5 to about 6.9, in which methadone is orally bioavailable through oral mucosal tissue at a rate of about 34%. However, the oral bioavailability of methadone via oral mucosa increases as the pH of the oral environment increases above this range. Particularly, bioavailability of oral transmucosal methadone has been found to be as high as about 50-75% where the oral cavity had a fluid pH of between about 7 and about 10. In one embodiment, the composition includes a pH buffer so as to maintain a pH in the oral cavity in the range from about 7 to about 10. In another embodiment, the composition includes a pH buffer so as to maintain a pH in the oral cavity in the range from about 8 to about 9, where the oral bioavailability of methadone has been shown to be as high as about 75%. Examples of suitable pH buffers include, without limitation, at least one of a phosphate buffer, a glycylglycine buffer, a carbonate buffer, a bicarbonate buffer, a tris buffer, a tartrate buffer, a borate buffer, an acetate buffer, and a maleate buffer. Combinations of buffers may be utilized to obtain the desired pH in the oral cavity.

The buffer should be present in an amount suitable to provide a desirable pH in an average patient's oral cavity upon administration of the composition and/or dissolution of the formulation in saliva or other oral cavity fluids. For example, generally amounts ranging from about 1% by weight to about 50% by weight should be suitable, depending upon the buffer. To buffer the oral solution, formed upon dissolution of the formulation, to a pH of about 6, a di-sodium phoshate(Na₂HPO₄)/citric acid buffer system (ratio of about 2:15:1) in about 2% by weight should be sufficient where the formulation comprises about 75% to about 90% of an oral dissolution agent. To buffer the oral solution to a pH of about 6.5, a phosphate buffer may be used in an amount resulting in about a 0.2 M concentration in the oral solution. To achieve a pH of about 8.5 in the oral cavity, a glycylglycine buffer can be used to provide a concentration of about 0.5 M in the oral solution. To achieve a pH of about 9.1, a carbonate-bicarbonate buffer system may be used in amounts resulting in about 1 M concentration in the oral solution. The amounts may be computed based upon saliva excretion rates in the oral cavity, and the specific methadone formulation and method of administration. Thus, formulating a pH buffer in a composition is one way of providing orally bioavailable methadone, absorbed across the oral mucosal tissue.

The amounts of the buffers, to be effective, generally depends upon the concentration of the fluids or solution in the oral cavity. As discussed in the Background, stimulated salivary glands, such as during chewing, biting, sucking, licking or other oral activity, excrete saliva at a rate of about 1-3 mls/minute. Thus, in as little as about 10-15 minutes, the total amount of saliva produced in the oral cavity is about 10-15 mls when stimulated. The amount of the pH buffer(s) should, therefore, be chosen according to the expected amount of the saliva in the oral cavity, the expected activity of the oral cavity during administration of the dosage formulation, the time required to dissolve a substantial portion of the dosage formulation, and the concentration of the resulting solution once the formulation dissolves in the oral cavity. Other known parameters, related to the effectiveness of a buffer to maintain a pH in a system, should also be considered when selecting the buffers and deciding the precise amounts to include in the composition.

The composition may further include additional pharmaceutical ingredients to provide desirable characteristics, such as aesthetically pleasing qualities, improved taste, and the like, to otherwise render the dosage formulation more likely to be administered by the patient. Examples of desirable ingredients include, without limitation, absorbants, colorants, flavorants, solvents and co-solvents, coating agents, direct compression excipients, disintegrants, glidants, lubricants, opaquants, polishing agents, suspending agents, sweetening agents, anti-adherents, binders, and capsule diluents. The ingredients may also include anti-fungal preservatives, anti-microbial preservatives, clarifying agents, emulsifying agents, antioxidants, levigating agents, plasticizers, surfactants, tonicity agents, viscosity increasing agents and combinations thereof. Examples of useful additives include, without limitation, propylene glycol, polyethylene glycol (PEG), orange, cherry, and strawberry flavors, stevia powder, and other commonly utilized ingredients.

The components of the composition may be formulated in any suitable orally dissolvable dosage form to deliver the methadone to the oral mucosal tissue. For example, suitable formulations include, without limitation, solid formulations such as a lozenge, a lollipop, a troche, a dragee, a chewable gum, a solid candy, a granular solid, a chewable tablet or pill, an orally dispersable tablet or pill, an orally dissolvable tablet, an orally dissolvable pill and an orally dissolvable capsule. In one embodiment, the composition is formulated as one of a lollipop and a lozenge, and includes an oral dissolution agent with the methadone. Alternatively, the formulation may be a liquid formulation, including, without limitation, a solution, a suspension, and an emulsion. For example, a liquid formulation, administrable as a spray, may be suitable, as may be a buffered suspension having the methadone and/or oral dissolution agent in fine granular form.

Such formulations may be prepared utilizing formulating procedures known in this art. For example, there are several ways to create a solid, orally dissolvable formulation, including, but not limited to, wet granulation, co-melt, spray-drying, freeze-drying, and the like. Particularly, solid formulations such as lozenges, solid candies, lollipops, or lozenges on a stick, and the like may be prepared utilizing such techniques, including wet granulation, co-melt, spray-drying, freeze-drying, and the like.

The process of wet granulation can be outlined as several steps: weighing and blending the ingredients of the composition in the presence of solvent(s), drying the mixture into solid, and milling the solid to proper size.

In the weighing and blending step of wet granulation, proper amounts of the oral dissolution agent(s), methadone, and solvent(s) are thoroughly mixed. Additional ingredients may be added to facilitate the mixing of the ingredients. The solvent(s) utilized should dissolve both the methadone and oral dissolution agent(s). The end result of this step is a finely blended mixture in which methadone and the dissolution agent are mixed at the molecular level. The mixture is then dried and generally ground to a powder so that it can be compressed into solid units. There are several ways to dry the wet granulation mixture depending on the mixture, the solvent, and the equipment. Milling and screening steps are usually used to ensure the proper particle size distribution for compression.

The solid formulation may also be made by a partial wet-granulation process. A formulation made using partial wet-granulation provides an opportunity for incorporating ionizable compounds, such as the hydrochloride salt of methadone. A partial wet granulation formulation can provide an oral environment that facilitates the methadone's dissolution therein, and methadone absorption through the oral mucosa. This method allows the pH in wet granulated particles to be buffered so that the methadone remains non-ionized, i.e., the granule maybe buffered to a high pH to prevent formation of quaternary salts of the methadone. Thus, upon dissolution in the oral cavity, the methadone is released and delivered as a free-based lipophilic form to the oral mucosal tissue, resulting in an effective rate of absorption.

There are many other processes for making solid formulations of methadone and the one or more oral dissolution agents, (i.e. processes that mix the methadone and dissolution agent(s) at the molecular level). The selection of the process will mainly depend on the methadone and dissolution agent(s). Lollipops, lozenges, and other suitable solid dosage formulations may be prepared by methods described in U.S. Pat. Nos. 4,671,953, 5,132,114, and 6,264,981, which disclosures are incorporated herein by reference in their entireties.

Similarly, liquid formulations are well known, and conventional procedures for their preparation are readily appreciated by those skilled in this art.

The composition is administered to the oral cavity of the patient. The patient may be instructed to orally dissolve the methadone-comprising dosage formulation, such as by biting it, chewing it, sucking on it, licking it, or merely storing it under the tongue or against the buccal tissue. Generally, during such masticatory activity, the patient's salivary glands are stimulated and excrete a suitable amount of saliva for dissolving or degrading the composition in the oral cavity. The oral dissolution agent(s) generally dissolve in the oral fluid thereby releasing methadone, and the pH buffer(s) where included, to “condition” the oral environment for improved methadone absorption. Thus, in this fashion, a substantial portion of the methadone may be delivered to the oral mucosal tissue for absorption therethrough.

During the patient's oral activity, the dissolving oral dissolution agents may expose the buffers from the formulation, or the buffers may themselves may dissolve in the patient's oral fluids. The buffers regulate the pH of the oral cavity to optimize absorption of the methadone delivered to the mucosa. For either of a solid or liquid formulation, the dissolution and disintegration rate may be controlled by appropriate selection of oral dissolution agents and/or pH buffers to control the release of methadone.

The therapeutic action of the methadone generally depends upon the contact time between the methadone and the oral mucosa surface, and on the rate of absorption into the blood. One factor affecting contact time is the dissolution rate of the formulation. However, another factor is how long the patient chooses to keep the formulation in his/her oral cavity. Once the formulation has dissolved, any methadone remaining in solution (oral fluid) and not yet absorbed will typically be swallowed, thereby ending further transmucosal methadone absorption. While the formulation generally remains in the mouth for about 10 to 15 minutes, this period will vary depending upon a number of factors. For example, how vigorously the dosage formulation is chewed, sucked, or licked will vary the time. In any event, many of the factors affecting the contact time, and therefore absorption and physiological effects of methadone, may generally be controlled by properly instructing the patient.

The compositions may be administered for providing to the patient any one, or a combination, of the many beneficial, therapeutic effects of methadone. For example, the composition may be administered to treat pain. In one embodiment, the composition is administered to treat pain attributed to one of cancer pain, neuropathic pain, chronic pain, acute pain, somatic pain, autonomic nervous system mediated pain, central pain, post-herpetic neuralgic pain, and combinations thereof. In another embodiment, the composition is administered to treat drug addiction, opioid tolerance, pathological itching, seizure, a sedative effect, a euphoric effect, an antitussive effect, an NMDA antagonistic effect, an opioid substitute for reducing opioid-induced constipation, and a reduction of at least one of catacholamine uptake, norepinephrine re-uptake and serotonine re-uptake, and a combination thereof. Generally, opioids induce constipation. However, methadone does so to a smaller degree than most other opioids, and therefore, switching to methadone therapy via the transmucosal route, should reduce the level of constipation experienced with other opioids.

The following groups of patients may by administered the compositions to obtain benefits from transmucosally absorbed methadone:
(1) patients in need of expedient pain relief, such as for breakthrough pain;
(2) patients with bowel obstruction;
(3) patients with nausea and/or vomiting;
(4) patients with dysphagia, mechanical or neurological pain;
   (5) patients who desire transmucosal opioids but do not get relief with transmucosal fentanyl;
(6) patients who desire transmucosal opioids but who are opposed to the taste of transmucosal fentanyl;
(7) patients currently on orally-ingested methadone (absorbed through the GI) but still in need of a breakthrough-pain analgesic;
(8) patients with opioid induced constipation. Particularly, since there is current evidence that oral methadone may be less constipating compared to other opioids (Daeninck and Bruera, 1999), and transmucosal methadone may further reduce the constipating effects because of the bypassing of the Gltract;
(9) patients suffering from the “addiction” stigma associated with methadone use, at which time the transmucosal methadone formulation should be used as an analgesic only;
(10) patients wishing to have the same drug as a long-acting and as a PRN. In such as case, polypharmacy and/or multiple prescription can be avoided;
(12) patients having “sensitive stomachs” to the GI effects of drugs orally ingested because the transmucosal methadone formulation would provide “rest for the stomach”; and
(13) patients that may or may not be able to properly maintain the lollipop or lozenge in the mouth. With fentanyl, the swallowed portion is wasted, but with methadone the swallowed portion is also absorbed.

While the purpose of administration and the desired therapeutic effect generally determines the methadone dosage administered, many additional factors should also be considered. For example, patient characteristics such as age, size, weight, gender and medical history, as well as the particular dosage formulation should be considered. Dosages in the range from about 0.5 mg to about 6 mg are generally effective for children, while higher doses in the range from about 2 mg to about 25 mg, for example, are generally effective for adults. Suitable doses of methadone for use in opioid naive patients include about 0.5 mg to about 2 mg. This dosage range may increase with practically no ceiling for treatment of opioid tolerant patients. Higher ranges may be useful on patients who are opioid tolerant having been previously treated with high doses of morphine and/or other opioids and were converted to methadone. Dosages should also be administered in accordance with a physician's approval, where appropriate.

To adequately treat the patient, the composition of the invention may be administered in conjunction with a second pharmaceutical composition. The second, co-administered composition may also comprise methadone, but in a formulation wherein the methadone is absorbed into the blood circulation in a substantially non-transmucosal route.

The invention will be further appreciated in the light of the following example.

### EXAMPLE

A candy matrix or base for the methadone is prepared by dissolving 50 gm of sucrose in 50 gm water and heating the solution to about 240° F. About 40 gm of corn syrup having a dextrose equivalent of about 42 units, and a high maltose content (about 30-35%) is added, and the mixture is heated at about 300° F. to reduce the water content by about 3%. After re-cooling the candy mass to about 240° F., suitable oil flavoring agents, such as cherry or lemon, are added. A solution of methadone hydrochloride at room temperature(about 100 mg) dissolved in about 10 ml of sterile water is gently added to the cooling candy mass at about 225° F. The solution is then poured into suitably shaped molds such as circular molds, bullet-shaped molds and the like, lubricated with vegetable oil to prevent sticking, having about a 6 cm³ capacity, and a wax-coated compressed paper stick is inserted into the base of each mold. The mold is allowed to set, and the candy mass allowed to harden. This process makes about 20 lollipops, each lollipop containing about 3.5 mg to about 4.0 mg of methadone equivalent.

The methadone may be included in the solution in amounts sufficient to provide 5 mg, 10 mg, and higher mg dosages, such as up to about 40 mg, of methadone per lollipop. Also, the lollipops may be produced by combining or physically mixing a sorbitol solution (70% sorbitol by weight in USP grade water), about 20% propylene glycol, methadone hydrochloride in an amount sufficient to provide 2 mg of methadone per lollipop. Sodium saccharin USP, flavoring agent(s), and stevia powder may further be included. The specific ingredient amounts may vary depending upon the desired characteristics of the lollipop. For example, the pH can be adjusted to a desired pH, such as about 8.5, by addition of sufficient amount of sodium hydroxide, advantageously in a solution. The lollipops may be small and have a weight ranging from about 2 to about 10 gm each.

By virtue of the foregoing, there are provided pharmaceutical compositions comprising methadone for transmucosal delivery in the oral cavity. The compositions provide safer and more effective analgesic relief over other formulations of methadone. Particularly, transmucosal delivery in the oral cavity avoids the local irritation otherwise experienced with the subcutaneous and intranasal delivery of methadone, and the cardiotoxicity of current parenteral methadone preparations. Thus, the invention provides non-parenteral, fast-acting formulations of methadone having clinical advantages, particularly for patients requiring rapid onset of sustained analgesia and requiring analgesia comparable to that obtainable with parenteral methadone, without the need for discomforts and problems related to intravenous access. The compositions further provide improved analgesia, with a longer duration of action, over other commonly administered opioids, and in particularly over fentanyl.

With increased bioavailability via the transmucosal route, smaller dosages of methadone are required for desirable therapeutic effects. Accordingly, the compositions provide methadone's desirable mu receptor agonist and NMDA receptor antagonist effects in a shorter period of time, and with minimal, if any, opioid tolerance and without the side effect profile of many of the other opioid analgesics.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative apparatus and method, and illustrated examples described. Accordingly, departures may be made from such details without departing from the scope or spirit of Applicants' general inventive concept.

1. A pharmaceutical composition comprising a dosage formulation including an oral dissolution agent, a buffer and methadone, said buffer effective to buffer the composition to a pH of at least about 6 during absorption of the methadone through the oral mucosa.

2. The composition of claim 1 wherein the composition is buffered to a pH in the range from about 7 to about 10 during absorption of the methadone through the oral mucosa.

3. The composition of claim 1 wherein the buffer is selected from the group consisting of a phosphate buffer, a glycylglycine buffer, a carbonate buffer, a bicarbonate buffer, a tris buffer, a tartrate buffer, a borate buffer, an acetate buffer, a maleate buffer and combinations thereof.

4. The composition of claim 1 in a solid formulation selected from a lozenge, a lollipop, a troche, a dragee, a chewable gum, a solid candy, a granular solid, a chewable tablet, an orally dispersable tablet, an orally dissolvable tablet, an orally dissolvable pill and an orally dissolvable capsule.

5. The composition of claim 1 in a liquid formulation selected from a solution, a suspension, a paste and an emulsion.

6. The composition of claim 1 containing at least about 0.5 mg of methadone per dose.

7. The composition of claim 1 containing methadone in a weight range from about 2 mg to about 50 mg per dose.

8. The composition of claim 1 wherein the oral dissolution agent is selected from the group consisting of acacia, alginic acid, carbomer, carboxymethylcellulose, calcium, carboxymethylcellulose sodium, microcrystalline cellulose, dextrates, dextrin, dextrose, methyl cellulose, ethyl cellulose, fructose, gelatin, guar gum, hydroxyethyl cellulose, hydroxypropyl cellulose, hydroxypropyl methylcellulose, lactitol, lactose, lecithin, maltodextrine, mannitol, poloxamer, polyethylene glycol, polymethacrylates, poly oxyethylene alkyl ethers, polyvinyl alcohol, propylene glycol alginate, sodium alginate, sodium ascorbate, sodium starch glyolate, sodium saccharin, sorbitol, starch, pregelatinized starch, sucrose, tragacanth, trimethylglycine, xanthan gum, xylitol, zein, and combinations thereof.

9. A pharmaceutical composition comprising a dosage formulation including an oral dissolution agent, a buffer, and at least about 0.5 mg methadone, the buffer effective to buffer the composition to a pH in the range from about 7 to about 10 during absorption of methadone through the oral mucosa.

10. The composition of claim 9 wherein the composition is buffered to a pH in the range from about 8 to about 9 during absorption of methadone through the oral mucosa.

11. The composition of claim 9 wherein the buffer is selected from the group consisting of a phosphate buffer, a glycylglycine buffer, a carbonate buffer, a bicarbonate buffer, a tris buffer, a tartrate buffer, a borate buffer, an acetate buffer, a maleate buffer and combinations thereof.

12. The composition of claim 9 in a solid formulation selected from the group consisting of a lozenge, a lollipop, a troche, a dragee, a chewable gum, a solid candy, a granular solid, a chewable tablet, an orally dispersable tablet, an orally dissolvable tablet, an orally dissolvable pill and an orally dissolvable capsule.

13. The composition of claim 9 containing methadone in a weight range from about 2 mg to about 50 mg per dose.

14. The composition of claim 9 wherein the formulation is one of a lollipop and a lozenge and contains methadone in a weight range from about 2 mg to about 10 mg per dose.

15. The composition of claim 9 wherein the oral dissolution agent is selected from the group consisting of acacia, alginic acid, carbomer, carboxymethylcellulose, calcium, carboxymethylcellulose sodium, microcrystalline cellulose, dextrates, dextrin, dextrose, methyl cellulose, ethyl cellulose, fructose, gelatin, guar gum, hydroxyethyl cellulose, hydroxypropyl cellulose, hydroxypropyl methylcellulose, lactitol, lactose, lecithin, maltodextrine, mannitol, poloxamer, polyethylene glycol, polymethacrylates, poly oxyethylene alkyl ethers, polyvinyl alcohol, propylene glycol alginate, sodium alginate, sodium ascorbate, sodium starch glyolate, sodium saccharin, sorbitol, starch, pregelatinized starch, sucrose, tragacanth, trimethylglycine, xanthan gum, xylitol, zein, and combinations thereof.

16. A pharmaceutical composition comprising methadone and an oral dissolution agent in a dosage formulation selected from one of a lollipop and a lozenge for oral administration of the methadone.

17. The composition of claim 16 further comprising a pH buffer in an amount sufficient to provide a pH in the range from about 6 to about 10 in the oral cavity.

18. The composition of claim 16 further comprising a pH buffer in an amount sufficient to provide a pH in the range from about 8 to about 9 in the oral cavity.

19. The composition of claim 17 wherein the pH buffer is selected from the group consisting of a phosphate buffer, a glycylglycine buffer, a carbonate buffer, a bicarbonate buffer, a tris buffer, a tartrate buffer, a borate buffer, an acetate buffer, a maleate buffer and combinations thereof.

20. The composition of claim 16 wherein the an oral dissolution agent is selected from the group consisting of acacia, alginic acid, carbomer, carboxymethylcellulose, calcium, carboxymethylcellulose sodium, microcrystalline cellulose, dextrates, dextrin, dextrose, methyl cellulose, ethyl cellulose, fructose, gelatin, guar gum, hydroxyethyl cellulose, hydroxypropyl cellulose, hydroxypropyl methylcellulose, lactitol, lactose, lecithin, maltodextrine, mannitol, poloxamer, polyethylene glycol, polymethacrylates, poly oxyethylene alkyl ethers, polyvinyl alcohol, propylene glycol alginate, sodium alginate, sodium ascorbate, sodium starch glyolate, sodium saccharin, sorbitol, starch, pregelatinized starch, sucrose, tragacanth, trimethylglycine, xanthan gum, xylitol, zein, and combinations thereof.

21. The composition of claim 16 containing at least about 0.5 mg of methadone per dose.

22. The composition of claim 16 containing methadone in a weight range from about 2 mg to about 10 mg per dose.

23. A method of transmucosally administering methadone to a patient, the method comprising:
providing to the patient a pharmaceutical composition comprising an orally dissolvable solid dosage formulation including methadone and a buffer effective to buffer the oral cavity to a pH of at least about 6 for oral transmucosal absorption of a therapeutically effective amount of the methadone; and
instructing the patient to orally dissolve the composition to transmucosally administer the methadone to the patient.

24. The method of claim 23 further comprising delivering the methadone to the blood circulation of the patient by absorption through the oral mucosal tissue.

25. The method of claim 23 wherein the patient is provided one of a solid formulation selected from a lozenge, a lollipop, a troche, a dragee, a chewable gum, a solid candy, a granular solid, a chewable tablet, an orally dispersable tablet, an orally dissolvable tablet, an orally dissolvable pill and an orally dissolvable capsule, and a liquid formulation selected from a solution, a suspension, and an emulsion.

26. The method of claim 23 wherein the methadone is administered to treat pain.

27. The method of claim 23 wherein the methadone is administered to provide at least one of an analgesic effect, a sedative effect, a euphoric effect, an antitussive effect, an NMDA antagonistic effect, an opioid substitute for reducing opioid-induced constipation, and a reduction of at least one of catacholamine uptake, norepinephrine re-uptake and serotonine re-uptake, in the patient's body.

28. The method of claim 23 wherein the methadone is administered to treat drug addiction, opioid tolerance, pathological itching, seizure, and a combination thereof.

29. A method of treating pain in a patient, the method comprising:
administering to the patient a pharmaceutical composition comprising an orally dissolvable solid dosage formulation including methadone and a buffer effective to buffer the oral cavity to a pH of at least about 6 for oral transmucosal absorption of a therapeutically effective amount of the methadone; and
delivering the methadone to the oral mucosal tissue for transmucosal absorption into the blood circulation to treat pain in the patient.

30. The method of claim 29 further comprising treating the patient for drug addiction, opioid tolerance, pathological itching, seizure, a sedative effect, a euphoric effect, an antitussive effect, an NMDA antagonistic effect, an opioid substitute for reducing opioid-induced constipation, and a reduction of at least one of catacholamine uptake, norepinephrine re-uptake and serotonine re-uptake, and a combination thereof in the patient's body.

31. The method of claim 29 wherein the pain is attributed to one of cancer pain, neuropathic pain, chronic pain, acute pain, somatic pain, autonomic nervous system mediated pain, central pain, post-herpetic neuralgic pain, and combinations thereof.

32. The method of claim 29 wherein the formulation administered is one of a solid formulation selected from a lozenge, a lollipop, a troche, a dragee, a chewable gum, a solid candy, a granular solid, a chewable tablet, an orally dispersable tablet, an orally dissolvable tablet, an orally dissolvable pill and an orally dissolvable capsule, and a liquid formulation selected from a solution, a suspension, and an emulsion.

33. The method of claim 29 further comprising administering the composition as needed to treat the pain.

34. The method of claim 29 further comprising administering to the patient a second pharmaceutical composition comprising methadone in a formulation wherein the methadone is absorbed into the blood circulation in a substantially non-transmucosal route.

35. A method of treating pain in a patient, the method comprising:
administering to the patient a pharmaceutical composition comprising methadone and an oral dissolution agent in a dosage formulation selected from one of a lollipop and a lozenge for oral administration of the methadone to treat pain in the patient.

36. The method of claim 35 further comprising delivering the methadone in the oral cavity of the patient for oral transmucosal absorption of the methadone into blood circulation.

37. The method of claim 35 further comprising treating the patient for drug addiction, opioid tolerance, pathological itching, seizure, a sedative effect, a euphoric effect, an antitussive effect, an NMDA antagonistic effect, an opioid substitute for reducing opioid induced constipation, and a reduction of at least one of catacholamine uptake, norepinephrine re-uptake and serotonine re-uptake, and a combination thereof in the patient's body.

38. The method of claim 35 wherein the pain is attributed to one of cancer pain, neuropathic pain, chronic pain, acute pain, somatic pain, autonomic nervous system mediated pain, central pain, post-herpetic neuralgic pain, and combinations thereof.

39. The method of claim 35 further comprising administering the composition as needed to treat the pain.

40. The method of claim 35 further comprising administering to the patient a second pharmaceutical composition comprising methadone in a formulation wherein the methadone is absorbed into the blood circulation in a substantially non-transmucosal route.
